(19) United States
(12) Patent Application Publication
(10) Pub. No.: US 2014/0283844 A1
(43) Pub. Date: Sep. 25, 2014

(54) Intra-Uterine Contraceptive Device

(71) Applicant: Sujoy Kumar GUHA, (US)

(72) Inventor: Sujoy Kumar Guha, New Delhi (IN)

(21) Appl. No.: 14/354,681

(22) PCT Filed: Oct. 25, 2012
(86) PCT No.: PCT/IN2012/000704

§ 371 Date: Apr. 28, 2014

(30) Foreign Application Priority Data
Oct. 28, 2011 (IN) 3084/DEL/2011

(51) Int. Cl. A61F 6/14; A61K 33/34; A61F 6/18; A61B 19/00

(52) U.S. Cl. CPC A61F 6/142; A61B 19/54; A61K 33/34; A61F 6/18; A61B 2019/5466
USPC 128/833; 128/839

(57) ABSTRACT

The present invention relates to an improved intra-uterine contraceptive device (IUCD) characterized by comprising polymer based material 1, and flexible structure 2, wherein the flexible structure 2 comprises one or more of tubelets 3, wherein the tubelets 3 are interconnected by connecting means 4, wherein the tubelets 3 are provided with a pulling means 5 on one end, wherein the tubelets 3 have perforations in the form of holes 6, and wherein the tubelets 3 have both sides 7, 8 open, and said sides are in sloping shape, wherein the tubelets 3 are arranged in the form of a chain, and wherein the combination of said polymer based material 1 and said flexible structure 2 is injectable or implantable in the uterine cavity. In one embodiment it relates to flexible structure referred to as intra-uterine device (IUD).

### CROSS-REFERENCE TO RELATED APPLICATIONS

This application is a filing under 35 U.S.C. 371 of International Application No. PCT/IN2012/000704 filed on Oct. 25, 2012, entitled “An Improved Intra-Uterine Contraceptive Device,” which claims priority to Indian Patent Application No. 3084/DEL/2011 filed on Oct. 28, 2011, which applications are incorporated by reference herein in their entirety.

### FIELD OF THE INVENTION

The present invention relates to an improved intra-uterine contraceptive device.

Particularly it relates to an intra-uterine contraceptive device (IUCD) which is capable of adapting to the changes in shape of the uterine cavity, and is capable of staying in the uterine cavity for longer duration, and thereby resisting expulsion from the uterine cavity, and is also capable of being removed from the uterine cavity without requiring invasive means.

More particularly it relates to an intra-uterine contraceptive device (IUCD) which is capable of adapting to the changes in shape of the uterine cavity, and is capable of staying in the uterine cavity for longer duration, and thereby resisting expulsion from the uterine cavity, and is also capable of being removed from the uterine cavity without requiring invasive means, and additionally is capable of not only causing contraceptive effect, but is also capable of causing antimicrobial effect, and thereby combating exogenous and endogenous infections.

Even more particularly it relates to an intra-uterine contraceptive device (IUCD) which is capable of adapting to the changes in shape and form of the uterine cavity, and is capable of staying in the uterine cavity for longer duration, and thereby, capable of resisting expulsion from the uterine cavity, and is also capable of being removed from the uterine cavity without requiring invasive means, and additionally is capable of not only causing contraceptive effect, but is also capable of causing antimicrobial effects, and thereby, combating exogenous and endogenous infections, and hence, lowering possibilities of infections including but not limited to sexually transmitted diseases including Acquired Immunodeficiency Syndrome (AIDS), and is capable of achieving these advantages without causing any stress in uterine cavity and/or on the uterine wall, and hence, without causing any trauma and consequent pain to the living being.

### BACKGROUND OF THE INVENTION

The intra-uterine contraceptive device (IUCD) is a female fertility control device implanted in the uterus to achieve contraception. A common adverse side effect of presently known IUCDs is that these are prone to cause cramping pain in the lower abdominal region and back pain, and even at times cause abnormal bleeding in-between menstrual periods due to their fixed form of structure. These adverse side effects limit adoption of known IUCDs by females in the reproductive age, and if adopted then often lead to the demand for early removal thereof.

The known IUCDs traumatize the lining of the internal cavity of the uterus and make the site susceptible to infection where the infective organism may be exogenous or endogenous from the vagina.

It has been observed that one important underlying cause to the adverse side effects of the known IUCDs is the mismatch between the shape of the IUCD and the uterine cavity, and incapability of the known IUCDs to adapt to the shape and form of the uterine cavity.

The known IUCDs are of a certain “fixed” shape and size, as for example the Copper T, which may be flexible in nature, but does not adapt to the dynamic variations in shape and form of the uterine cavity. It is known that shape and form of the uterine cavity differs from individual to individual, and the uterine cavity of a particular individual also changes in size, shape and form from time to time on account of contractions of the uterine muscles.

It has been observed that the presently known IUCDs even when made from a flexible material do not have the capability of continually adapting to shape and form of the uterine cavity. Therefore, the known IUCDs suffer from problems of causing considerable mechanical stresses in the uterine cavity and on the wall of the uterine cavity. Even the known IUCDs tend to probe into the wall of the uterine cavity. These stresses are known to activate nerves and pain sensors and also traumatize the uterine wall leading to bleeding, and thereby, the injured regions become prone to pick up further infections.

The problems of known IUCDs have been overcome by providing contraceptives which convert to a solid or semi-solid form within the uterine cavity. However, some such contraceptives suffer from the problems of early expulsion or removal. Other contraceptives do not get removed early but they set so firmly that it requires an invasive means to remove it. Therefore, one advantage is achieved at the cost of another advantage.

A fixed form IUCD coated with an antimicrobial compound which is typically a complex of styrene maleic anhydride (SMAn) and dimethyl sulfoxide (DMSO) was developed, wherein the coating of antimicrobial compound is a thin layer. However, it has been found that even this IUCD having thin layer of coating of antimicrobial material over the fixed form of IUCD establishes the antimicrobial zone, but only over a small zone around the IUCD, and not in the entire uterine cavity. Another drawback of this fixed form of IUCD is that there is no release of antimicrobials from the IUCD coating to make the walls of the uterine cavity antimicrobial and infection resistant.

The prior art WO 2011/080164 A1 discloses an intrauterine device comprising a suture thread, supporting the active component and an anchor to be inserted in the wall of the uterus with a specially designed applicator or inserter in order to secure the IUD/IUS in the uterine cavity, wherein the anchor consists of a knot made at one end of the thread, and a tiny metal tube, preferably of stainless steel, is threaded onto the thread and fixed immediately below or above the anchoring knot to make the anchor visible on ultrasound and/or x-ray, and the anchoring knot may be provided with a tightly fitting ogival, bell or cone-shaped cap consisting of a biodegradable polymer for ease of insertion and retention, and thereby, to reduce risk of expulsion in cases where the uterine wall is soft, and this polymer degrades in weeks or a few months. There is neither any disclosure nor any teaching in D1 that the polymer based material may comprise a contraceptive polymer composition, and may be combined with a flexible device comprising one or more of tubelets having perforations in the form of holes have both ends being open, and said ends having sloping shape, and wherein the combination of said polymer based material and said flexible structure is injectable or implantable in the uterine cavity.

The prior art EP 0 673 629 A1 discloses an intrauterine contraceptive device comprising at least two flexible arms, extending from a central point, provided with a flexible thread, means acting as contraceptive and means for promoting retention of the contraceptive device in the uterus, wherein the tips of the flexible arms are massive and directed upward toward the fundus, whereby the retention means are cavities provided from the central point to about the massive tips along the front and back of the arms, while the flexible thread is surrounded by the means acting as contraceptive, while the thread is provided with a holding device for the means acting as contraceptive.

### NEED OF THE INVENTION

Therefore, there is a need of providing an improved intra-uterine contraceptive device (IUCD) which is not only capable of adapting to the changes in shape and form of the uterine cavity, but is also capable of staying in the uterine cavity for a longer duration, and additionally is also capable of causing long-term contraceptive effect, and causing antimicrobial effects not only in the close proximity of the IUCD, but also within the entire uterine cavity, and on and around inner walls thereof, and can still be removed from the uterine cavity without requiring invasive means to restore fertility.

### Problem to be Solved by the Invention

Therefore, the present invention aims to solve above-described problems of the prior art by providing an improved intra-uterine contraceptive device (IUCD) which is not only capable of adapting to the changes in shape and form of the uterine cavity, but is also capable of staying in the uterine cavity for a longer duration, and additionally is also capable of causing long-term contraceptive effect, and causing antimicrobial effects, and thereby combating exogenous and endogenous infections not only in the close proximity of the IUCD, but also within the entire uterine cavity, and on and around inner walls thereof, and hence, lowering possibilities of infections including but not limited to sexually transmitted diseases including AIDS, and can still be removed from the uterine cavity without requiring invasive means to restore fertility, and is capable of achieving these advantages without causing any stress in uterine cavity and/or on the uterine wall, and hence, without causing any trauma and consequent pain to the living being.

### Objects and Advantages of the Invention

Accordingly, the main object of the present invention is to provide an improved intra-uterine contraceptive device (IUCD) which is not only capable of adapting to the changes in shape and form of the uterine cavity, but is also capable of staying in the uterine cavity for a longer duration and resist expulsion from the uterine cavity.

Another object of the present invention is to provide an improved intra-uterine contraceptive device (IUCD) which is additionally capable of causing long-term contraceptive effect.

Still another object of the present invention is to provide an improved intra-uterine contraceptive device (IUCD) which is additionally capable of causing antimicrobial effects not only in the close proximity of the IUCD, but also within the entire uterine cavity, and on and around inner walls of the uterine cavity, and hence, lowering possibilities of infections including but not limited to sexually transmitted diseases including AIDS.

Yet another object of the present invention is to provide an improved intra-uterine contraceptive device (IUCD) which can be removed from the uterine cavity without requiring invasive means to restore fertility.

This is also an object of the present invention to provide an improved intra-uterine contraceptive device (IUCD) which does not cause stress in uterine cavity and/or on the uterine wall, and therefore, does not cause trauma and consequent pain to the living being.

This is further an object of the present invention to provide an improved intra-uterine contraceptive device (IUCD) which is capable of developing, within the uterine cavity, antimicrobial mass or surface which makes inner walls of the uterine cavity antimicrobial and infection resistant, and hence capable of combating exogenous and endogenous infections.

The other objects and advantages of present invention will become more apparent from the following description when read in conjunction with the accompanying figures which are not intended to limit scope of present invention.

### BRIEF DESCRIPTION OF ACCOMPANYING FIGURES

It may be noted that the accompanying figures are not to scale. These are only illustrative of features of present invention.

FIG. 1 is a schematic representation of improved intra-uterine contraceptive device (IUCD) of the present invention with its implantation means (inserter) in accordance with one of the embodiments of the present invention.

FIG. 2 is a schematic representation of intra-uterine device (IUD) chain of the improved intra-uterine contraceptive device (IUCD) in accordance with one of the embodiments of the present invention. Not shown in the figure, however, the tubelets of the IUD chain are filled with the polymer based material, when it is inserted or implanted in the uterus.

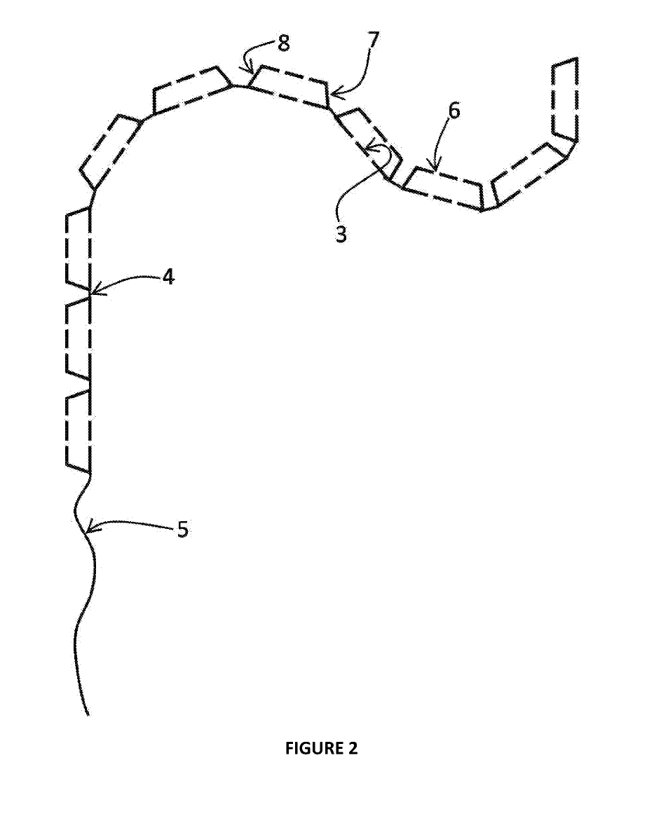

FIG. 3 is a schematic representation of the uterus with the improved intra-uterine contraceptive device (IUCD) in accordance with one of the embodiments of the present invention.

FIG. 4 is a prototype of intra-uterine device (IUD) chain of the improved intra-uterine contraceptive device (IUCD) in accordance with one of the embodiments of the present invention.

FIG. 5 illustrates that during in-vitro studies, the tubelets of the IUCD in accordance with one of the embodiments of the present invention show that polymer based material oozes out of the perforation holes after swelling of the polymer based material.

FIG. 6 illustrates that from the perforations of the tubelets of the IUCD in accordance with one of the embodiments of the present invention, the polymer based material oozes out and links with the implant masses and forms link bridges.

FIG. 7A illustrates X-Ray of the cadaver goat pelvis with a prototype IUCD in accordance with one of the embodiments of the present invention being retained within the cadaver goat uterus.

FIG. 7B illustrates X-Ray of the cadaver goat pelvis with a prototype IUCD in accordance with one of the embodiments of the present invention being retained within the cadaver goat uterus along with polymer based material.

FIG. 7C illustrates X-Ray of the cadaver goat pelvis after removal of a prototype IUCD in accordance with one of the embodiments of the present invention, but the polymer based material is still retained within the cadaver goat uterus.

FIG. 8 illustrates pelvic ultrasonogram with a rectal probe in a live goat showing a prototype IUCD in accordance with one of the embodiments of the present invention, wherein the location of the tubelets in the image indicates a bent non-linear distribution of the tubelets of the IUCD in accordance with one of the embodiments of the present invention.

FIG. 9 illustrates X-Ray of the pelvis of a live goat with a prototype IUCD in accordance with one of the embodiments of the present invention being retained within the goat uterus. It may be noted that in FIG. 9, the polymer based material is not seen, however, when X-Ray is examined in the X-Ray viewer, it was found that the IUCD is retained within the cadaver goat uterus along with polymer based material.

### SUMMARY OF THE INVENTION

It is understood from the foregoing description that absence of a free form IUCD is posing risks to females and is limiting the acceptance of IUCDs as a contraceptive.

With aim to overcome above-described problems of the prior art by providing a free form IUCD which can be acceptable to females, the inventor has found, by his preliminary experiments on cadaver goats as well as live goats that if a contraceptive is injected into the uterine cavity along with a flexible structure which is also capable of releasing the antimicrobial contraceptive within the uterus, the combination of the contraceptive and the flexible structure [forming improved intra-uterine contraceptive device (IUCD) of the present invention], surprisingly and unexpectedly, adapts to the changes in shape and form of the uterine cavity from time to time and also stays in the uterine cavity for a longer duration. Further the endometrium of the uterus acquires antimicrobial property.

The inventor has further found that said IUCD can be removed without requiring invasive means.

It may be noted that these experiments have been carried out on the cadaver goats as well as live goats.

The inventor, by in-vitro and in-vivo studies, has found that the IUCD comprising a polymer based material (the contraceptive or antimicrobial contraceptive material) and the flexible structure is, additionally, capable of causing long-term contraceptive effect due to formation of compact mass type structure of the contraceptive and the flexible structure within the uterine cavity.

The inventor, by cadaver and live goat studies, has further found that the IUCD comprising a polymer based material (the contraceptive or antimicrobial contraceptive material) and the flexible structure is, additionally, capable of causing antimicrobial effects not only in the close proximity of the IUCD, but also within the entire uterine cavity, and on and around walls thereof due to formation of an antimicrobial mass within the uterine cavity, and antimicrobial surface on and around the walls of the uterine cavity.

Accordingly, the present invention relates to an improved intra-uterine contraceptive device (IUCD) characterized by comprising a polymer based material and flexible structure, wherein the flexible structure comprises tubelets interconnected by connecting means, and is provided with a pulling means on one end, wherein the tubelets filled with a polymer based material have perforations in the form of holes, and have both sides open and in sloping shape, wherein the combination of polymer based material and flexible structure is injectable in the uterine cavity.

Accordingly, the present invention, in another embodiment, also relates to intra-uterine device (IUD) characterized by comprising a flexible structure, wherein the flexible structure comprises tubelets interconnected by connecting means, and is provided with a pulling means on one end, wherein the tubelets have perforations in the form of holes, and have both sides open and in sloping shape, wherein the flexible structure is injectable in the uterine cavity.

### DESCRIPTION AND PREFERRED EMBODIMENTS OF THE INVENTION

Now, referring to accompanying figures, which are not intended to limit scope of present invention, but have been incorporated merely to illustrate the invention, the present invention relates to an improved intra-uterine contraceptive device (IUCD) characterized by comprising
polymer based material 1, and
flexible structure 2,
wherein
the flexible structure 2 comprises
   one or more of tubelets 3,
   wherein the tubelets 3 are interconnected by connecting means 4,
   wherein the tubelets 3 are provided with a pulling means 5 on one end,
   wherein the tubelets 3 have perforations in the form of holes 6, and
   wherein the tubelets 3 have both sides 7, 8 open, and said sides are in sloping shape,
   wherein the tubelets 3 are arranged in the form of a chain, and
wherein the combination of said polymer based material 1 and said flexible structure 2 is injectable or implantable in the uterine cavity.

In accordance with another embodiment of the present invention, it also relates to an intra-uterine device (IUD) (or IUD chain) characterized by comprising
a flexible structure 2, comprising
   one or more of tubelets 3,
   wherein the tubelets 3 are interconnected by connecting means 4,
   wherein the tubelets 3 are provided with a pulling means 5 on one end,
   wherein the tubelets 3 have perforations in the form of holes 6, and
   wherein the tubelets 3 have both sides 7, 8 open, and said sides are in sloping shape,
   wherein the tubelets 3 are arranged in the form of a chain.

In accordance with one of the embodiments of the present invention, the IUD chain further comprises a polymer based material, that is, it is implanted or injected in the uterine cavity along with or in combination with a polymer based material 1.

In accordance with one of the embodiments of the present invention, the polymer based material is one which causes contraceptive effects.

In accordance with one of the embodiments of the present invention, the polymer based material is one which causes contraceptive effects and antimicrobial effects.

In accordance with one of the embodiments of the present invention, the polymer based material is one which causes antimicrobial effects.

In accordance with one of the embodiments of the present invention, the polymer based material comprises an antimicrobial contraceptive polymer, which is capable of releasing antimicrobial fragments of polymer which sets within the uterus in the form of dispersed, but linked varied shaped globules and, which bridges with masses of the same polymer emerging from tubelets of the flexible structure.

In accordance with one of the preferred embodiments of the present invention, the tubelets 3 are made of a polymer material, preferably of a semi-rigid polymer material, more preferably of a porous dimethyl sulfoxide resistant polymer, even more preferably of a polymer material selected from a group comprising a pure natural rubber and polytetrafluoroethylene (PTFE), and still more preferably of polytetrafluoroethylene (PTFE).

In accordance with one of the embodiments of the present invention, the flexible structure with perforated small tubelets is a free form structure, that is having no fixed structure.

In accordance with the present invention, the combination of the polymer based material and the tubular IUD chain comprising perforated small tubelets is an improved intra-uterine contraceptive device (IUCD) of the present invention, which places the polymer based material within the uterine cavity and retains it for a longer duration.

In accordance with one of the embodiments of the present invention, the connecting means of the IUD chain are flexible.

The sloping sides 7 and 8 of the tubelets 3 and flexibility of connecting means 4 of the IUD chain allows the (flexible) structure to be flexible and to change its shape and forms and to adapt to the changes in the shape and form of the uterine cavity from time to time.

In accordance with one of the preferred embodiments of the present invention, the polymer based material 1 comprises “polymer composition” and copper.

In accordance with one of the preferred embodiments of the present invention, the copper is in the form of closed copper tubelets.

In accordance with one of the preferred embodiments of the present invention, the closed copper tubelets are elongated.

In accordance with one of the preferred embodiments of the present invention, the closed copper tubelets are air filled.

The advantage of closed copper tubelets of present invention is that these provide structural strength to the IUCD of present invention.

Another advantage of closed copper tubelets of present invention is that these provide contrast for ultrasonic imaging of the IUCD of present invention.

In accordance with another preferred embodiment of the present invention, the polymer based material 1 comprises “polymer composition” and radio-opaque material.

In accordance with one of the preferred embodiments of the present invention, the radio-opaque material is selected from barium sulfate and iodine or combination thereof, which may be mixed with the polymer based material.

The advantage of radio-opaque material is to have convenient X-Ray imaging of the IUCD of present invention to detect its presence.

In accordance with one of the preferred embodiments of the present invention, the polymer based material comprises “polymer composition”, a copper in the form of closed copper tubelets and radio-opaque material.

In accordance with one of the embodiments of the present invention, the said “polymer composition” of the said “polymer based material 1” is selected from a group comprising contraceptive polymer comprising high molecular weight styrene maleic anhydride and dimethyl sulfoxide (DMSO) (U.S. Pat. No. 5,488,075); contraceptive polymer comprising a contraceptive polymer, a solvent medium, an electrically conducting material and a magnetic material (International Patent Application No. PCT/IN2000/000023 having PCT publication No. WO 2000/054746 claiming priority from the Indian Patent No. 233336, formerly Indian Patent Application No. 415/DEL/1999); and styrene maleic anhydride based synergistic formulation comprising styrene maleic anhydride having lower molecular weight and styrene maleic anhydride having higher molecular weight dissolved in DMSO (International Patent Application No. PCT/IN2009/000161 having PCT publication No. WO2009/113108 A2 claiming priority from the Indian Patent Application No. 618/DEL/2008).

In the contraceptive polymer comprising a contraceptive polymer, a solvent medium, an electrically conducting material and a magnetic material of the PCT publication No. WO 2000/054746, the contraceptive polymer is from the hydrogel class of polymers, particularly a mixture of styrene maleic anhydride copolymer and styrene maleic acid copolymer, and said solvent medium is dimethyl sulfoxide solvent, and said electrically conducting material is copper in its pure form essentially consisting of microsize particles and macrosize particles, and said magnetic material is iron in its pure form essentially consisting of microsize particles and macrosize particles.

In the styrene maleic anhydride based formulation comprising styrene maleic anhydride having lower molecular weight and styrene maleic anhydride having higher molecular weight dissolved in DMSO of the PCT publication No. WO2009/113108, the molecular weight of said SMA having lower molecular weight varies in the range from about 10000 to about 20000 and the molecular weight of said SMA having higher molecular weight varies in the range from about 60000 to about 100000.

The advantage of pulling means 5 provided on one end of the IUD chain of the present invention is to facilitate removal of the IUCD of present invention and additionally to confirm that the device is inside the uterus.

In accordance with present invention, the tubelets 3, the connecting means 4 and the pulling means 5 may be made from same polymer. In accordance with one of the preferred embodiments of the present invention, the said polymer is semi-rigid polymer material, preferably a porous dimethyl sulfoxide resistant polymer, more preferably is selected from a group comprising pure natural rubber and polytetrafluoroethylene (PTFE), and even more preferably is polytetrafluoroethylene (PTFE, trade name Teflon).

In accordance with one of the preferred embodiments of the present invention, the polymer based material may additionally comprise one or more of compounds selected from the group comprising spermicidal, ovicidal, antimicrobial, and antimicrobial fragment releasing compounds or combination thereof. The polymer based material is initially in the form of a viscous thick liquid.

In accordance with one of the preferred embodiments of the present invention, the polymer based material may additionally comprise one or more of compounds selected from the group comprising iron, copper, zinc and other materials or combination thereof to enhance structural strength and increase spermicidal, ovicidal and antimicrobial properties of the present device.

The base antimicrobial polymer also releases fragments which render the inner wall of the uterine cavity antimicrobial.

The tubelets of the IUD chain and the IUCD of the present invention release fragments of the polymer which set within the uterus in the form of dispersed but linked varied shaped globules and which bridge with polymer injected along with the IUD chain.

The polymer from the tubelets of the IUD chain and the polymer based material of the IUCD of the present invention releases fragments which render the inner walls of the uterine cavity antimicrobial.

The sloping shaped ends 7, 8 of the tubelets 3 of IUD chain have additional advantage of allowing neighboring tubelets to overlap each other without causing any obstruction to each other for adapting to changes in the shape and form of the uterine cavity from time to time.

The uterine cavity is bounded by an upper roof 21 and two sloping side walls 22 on each side. The communication between the vagina and the uterus is the cervical canal 23 which is narrow. The inside lining of the uterus roof and wall is the endometrium lining 24. The uterine cavity 25 is a flattened space in the antero-posterior direction. The mechanism of functioning of present invention is not fully established at present. It is believed that the polymer base material of the IUCD of the present invention on being pumped or injected or implanted into the uterine cavity reacts with water and the precipitating solution, which is pumped separately into the uterus to form a dispersed semisolid implant 26. The implant is in the form of dispersed masses of the polymer based material with interconnected space amongst the mass forms. These spaces form the passage for menstrual blood drainage at the time of menstruation. From the perforations of the tubelets of the IUD chain, the polymer based material oozes out and links with the implant masses forming link bridges 27. At various places in the implant there are dispersed air filled, elongated, closed copper tubelets 28. The surface of the copper tubelets is preferably made rough so that it helps to anchor together segments of the implant thereby giving structural strength and resistance to expulsion at the time of uterus contractions.

Accordingly, in accordance with one of the preferred embodiments of the present invention, the precipitating solution is pumped separately into the uterus after the IUCD of the present invention has been pumped or injected or implanted into the uterine cavity.

In accordance with one of the preferred embodiments of the present invention, the precipitating solution is acidic in nature.

In accordance with one of the preferred embodiments of the present invention, the precipitating solution is acidified saline.

The IUCD of the present invention can be implanted in the uterine cavity by any known means.

In accordance with one of the preferred embodiments of the present invention, the IUCD of the present invention is implanted in the uterus with the implantation means 9 comprising semi-rigid inserting means 10. The IUD chain is positioned within this inserting means 10 with the pulling means 5 emerging from one end of the inserting means. There is provided a pushing means 11 which can be pushed inside the inserting means. One side of the inserting means is provided with syringe connector 12, which is preferably tapered having an inbuilt one way valve 13 which is capable of allowing the thick liquid from the syringe 14 to flow into the inserting means but obstruct back flow. The nozzle of the syringe fits into the syringe connecter 12. The syringe 14 is provided with a pushing means (piston) 15 and the pushing means is connected to a thumb rest 16 with a connecting rod 17.

Accordingly, in one of the embodiments of the present invention, it relates to the intra-uterine contraceptive device (IUCD) of the present invention implantable in the uterus through an implantation means 9 comprising:
semi-rigid inserting means 10 provided with
i) a pushing means 11, and
ii) a syringe connector 12 having an inbuilt one way valve 13 which is capable of allowing the thick liquid from the syringe 14 to flow into the inserting means but obstruct back flow,
wherein the syringe 14 is provided with a pushing means (piston) 15, and
wherein the pushing means is connected to a thumb rest 16 with a connecting rod 17.

The nozzle of the syringe fits into the syringe connecter 12.

In accordance with one of the preferred embodiments of the present invention, the syringe connector 12 is preferably arranged in a tapered manner onto inserting means 10.

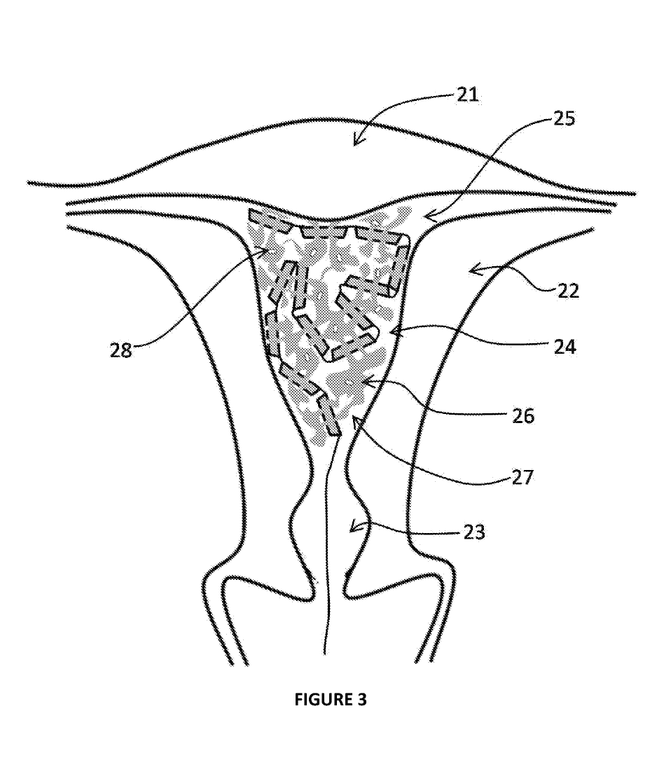

For positioning the IUCD of present invention into the uterus first the syringe containing the polymer based material which is in a highly viscous, thick liquid form is connected to the inserting means by pushing the syringe nozzle into the syringe connecter. Holding the inserting means vertical, the piston is pushed into the syringe to expel the polymer based material into the inserting means. The polymer based material passes through the tubelets of the IUD chain to fill the upper portion of the inserting means. Next the upper end of the inserter is inserted into the cervical canal of the uterus so that the open end of the inserter is just below the inner surface of the roof of the uterus. Then the piston is pushed upwards. At the same time the inserter is pulled downwards slowly and concurrently the inserter is turned to the left and right so that the open end of the inserter traverses a zig zag path within the uterine cavity. The pushing of the piston upwards expels the polymer based material stored in the upper segment of the inserter and also pushes out the tubelets of the IUD chain into the uterine cavity. Since the inserter upper open end is moved from side to side the IUD chain as it emerges out of the inserter occupies a zig zag form within the uterine cavity as shown in FIG. 3. The polymer based material thick liquid as it comes out of the open upper end of the inserter forms a zig zag of small islands of the material and not a blob in one spot. During this process backflow of the polymer based material out of the inserter is checked by the one-way valve 13.

Thereafter, the syringe 14 is disconnected from the inserter by withdrawing the nozzle end of the syringe from the syringe connecter. Next a syringe, not shown in the figure, containing a precipitating solution such as acidified saline is connected to the syringe connecter and small amount typically varying upto about 3 ml of the precipitating solution is pumped into the uterine cavity. After waiting for two minutes for polymer based material to react with the precipitating solution and undergo the initial phase of conversion to a semi solid mass, and the inserter is withdrawn from the uterus. The IUCD implantation procedure is then complete and the recipient female is free to walk away from the procedure table.

In the course of a couple of hours the precipitating chemical reaction of the polymer based material is completed. The implant becomes semi solid to some extent yet remains soft and deformable. Also the bridges 27 between the IUD chain tubelets and the main base material mass are formed. The implant, thereby, acquires structural strength to resist expulsion. The chemical reaction of precipitation concurrently leads to opening of some chemical bonds in the polymer based material and there is pH lowering and development of an electrical charge on the surface of the islands of polymer based material. The surface thereby acquires antimicrobial property. In time very small fragments of the base material come out of the implant bulk to enter the wall of the lining of the uterine cavity thus making the surface antimicrobial. In this manner a free form antimicrobial IUCD is established within the uterus, which is capable of resulting in contraception and infection protection.

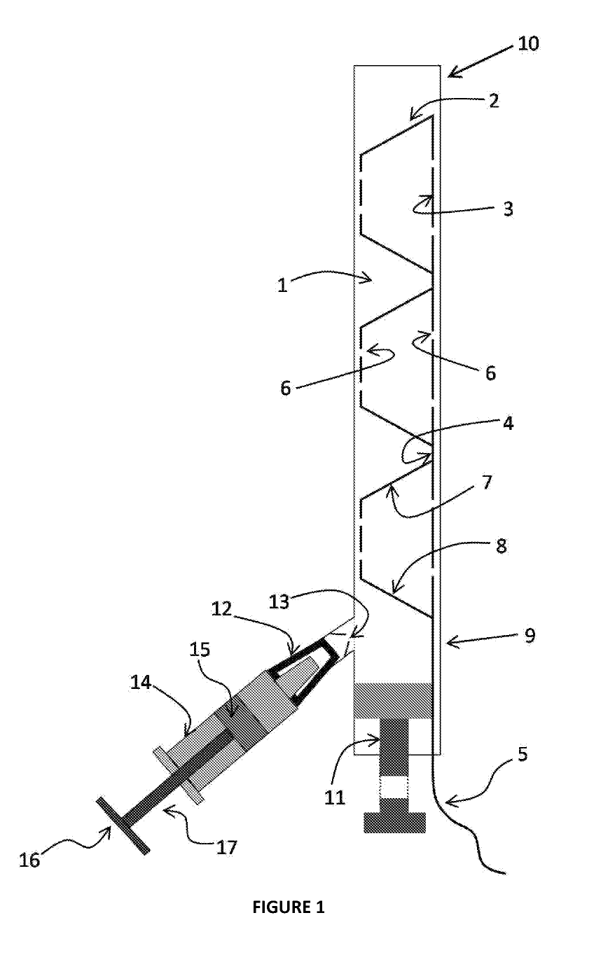

It may be noted that FIG. 1 shows IUD chain comprising three tubelets for diagrammatic convenience. In accordance with one of the embodiments of the present invention, the number of tubelets, preferably of length varying from about 2 to about 5 mm, varies upto 15, preferably from about 3 to about 15 depending upon the uterus size of the particular female recipient are placed within the inserting means.

In accordance with one of the preferred embodiments of the present invention, the inserting means 10 has length varying upto about 20 cm and diameter varying upto about 7 mm.

### EXPERIMENTAL STUDIES

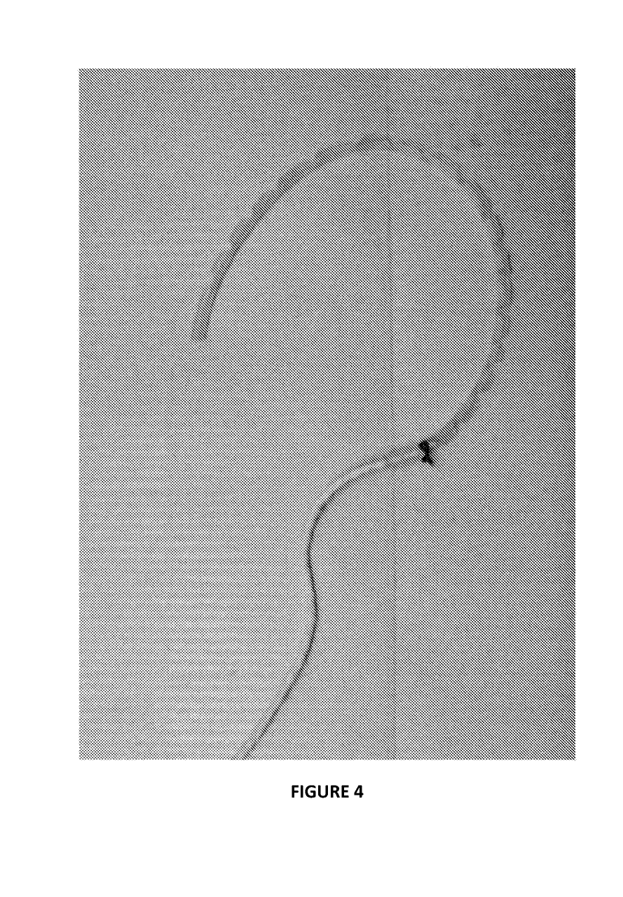

For the experimental studies, a prototype of intra-uterine device (IUD) chain of the improved intra-uterine contraceptive device (IUCD) was manufactured (FIG. 4).

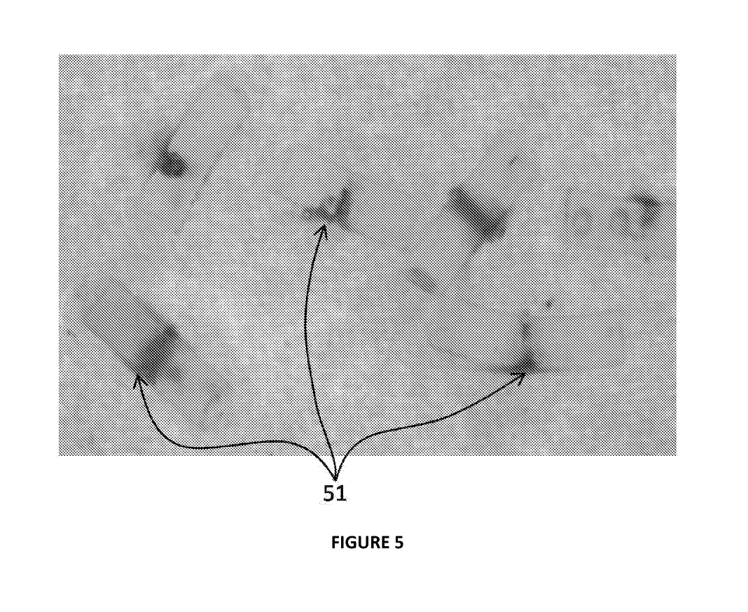

During the in-vitro studies, the tubelets of the IUCD have demonstrated that polymer based material oozes out of the perforation holes after swelling of the polymer as can be seen in FIG. 5 by numeral 51.

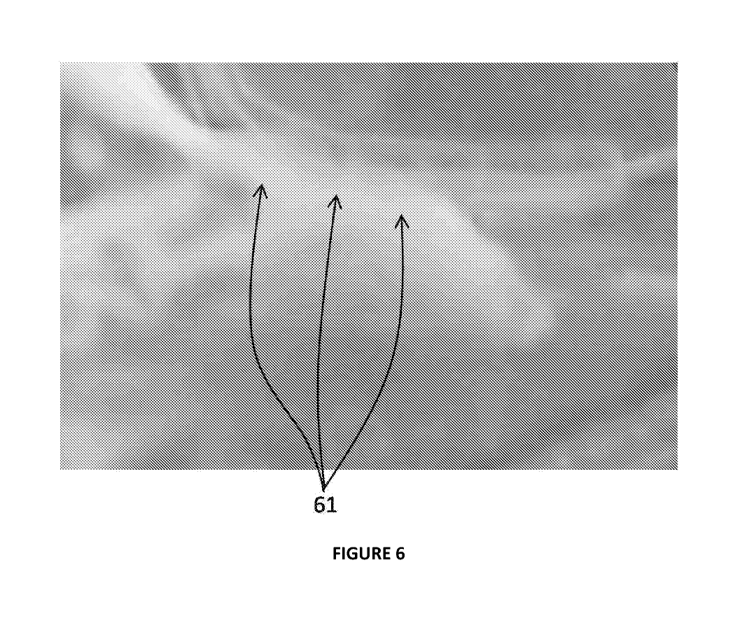

During the in-vitro studies, it has been found that from the perforations of the tubelets of the IUCD, the polymer based material oozes out and links with the implant masses and forms link bridges as can be seen in FIG. 6 by numeral 61.

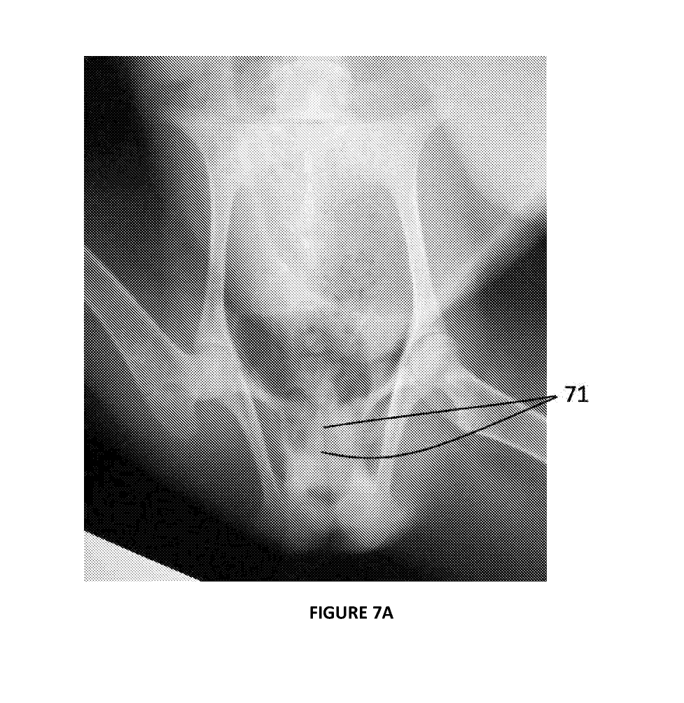

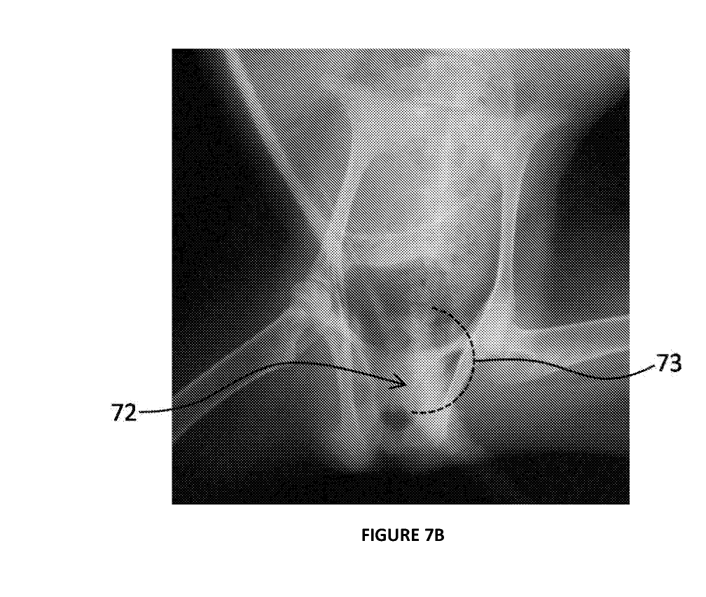

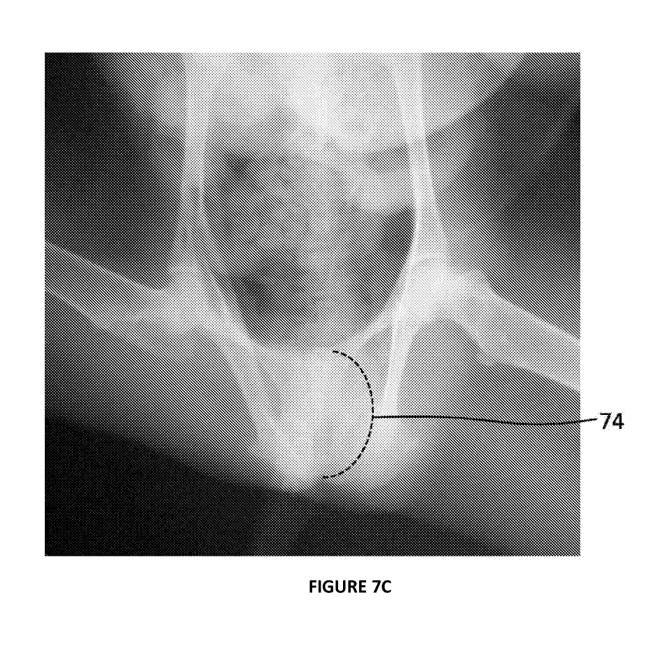

During the in-vivo studies on a cadaver goat it has been found that a prototype IUD once inserted by employing inserting means of the present invention remains within the cadaver goat uterus as can be seen in the X-Ray of the cadaver goat pelvis shown in FIG. 7A by numeral 71.

During the in-vivo studies on a cadaver goat it has also been found that a prototype IUCD once inserted by employing inserting means of the present invention remains within the cadaver goat uterus as can be seen in the X-Ray of the cadaver goat pelvis shown in FIG. 7B, wherein IUCD is shown by numeral 72 and polymer based material is shown by numeral 73.

During the in-vivo studies on a cadaver goat it has also been found that a prototype IUCD once inserted by employing inserting means of the present invention remains within the cadaver goat uterus along with polymer based material, and when the IUD of the IUCD of the present invention is withdrawn, it was found that the polymer based material still remains within the cadaver goat uterus as can be seen in the X-Ray of the cadaver goat pelvis shown in FIG. 7C by numeral 74.

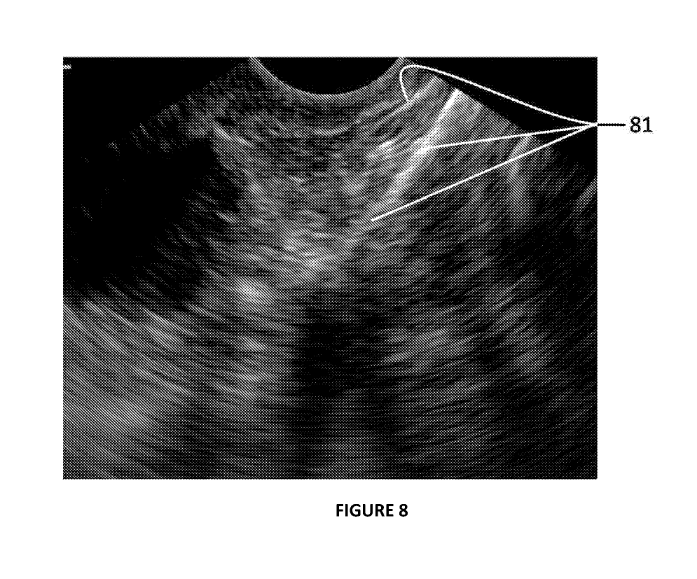

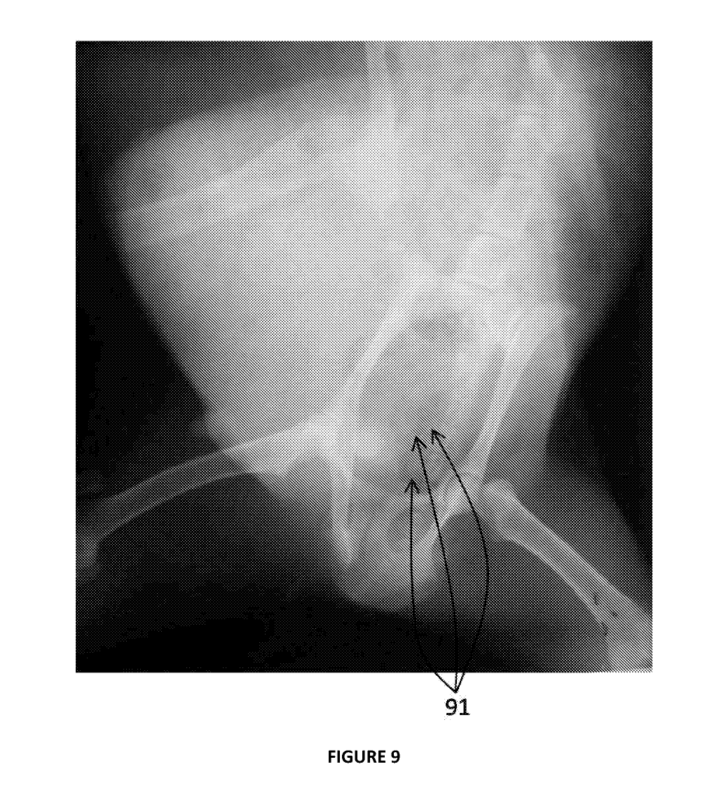

During the in-vivo studies on a live goat it has also been found that the prototype IUCD once inserted by employing inserting means of the present invention remains within the goat uterus (along with polymer based material) as can be seen in the pelvic ultrasonogram using a rectal probe on a live goat with shown in FIG. 8 by numeral 81, wherein the location of the tubelets in the image indicates a bent non-linear distribution of the tubelets of the IUCD, and in the X-Ray of the pelvis of a live goat shown in FIG. 9, wherein the IUCD is shown by numeral 91.

In proven fertile two live goats, the IUCD comprising polymer based material of present invention was inserted by employing inserting means of the present invention, which results in an IUCD comprising tubelets filled with polymer based material and surrounded with polymer based material. The animals were clinically found to be normal over a period of observation of about 4 months and retained the IUCD as confirmed by manual examination, pelvic ultrasonography [FIG. 8] and pelvic X-Ray [FIG. 9]. On mating with proven fertile male no pregnancy occurred as determined by clinical examination and ultrasonography over two months post-coital and supplementary urine examination. Therefore, the contraceptive efficacy of the IUCD of the present invention was established.

It can be seen from FIGS. 7A, 7B and 7C that the constituents of the prototype IUD chain of improved IUCD in accordance with one of the embodiments of the present invention occupies different positions. The goat uterus is somewhat straight, and hence, the difference in angulation of the constituents of the prototype IUD chain of IUCD of the present invention is not too much. In human beings with the pyriform shape and a broader uterus the angulations between tubelets will be more.

To confirm advantages of present invention, the in-vitro and in-vivo tests were conducted by employing IUCD manufactured in accordance with one of the preferred embodiments of the present invention, wherein the polymer based material fills the tubelets of the IUCD and also surrounds the tubelets of the IUCD, which acts as an uterine implant.

The in-vitro tests carried out as per known methods have confirmed that the IUCD of the present invention has antimicrobial action against a range of microorganisms including Bacteria—gram positive (Staphylococcus aureus) and gram negative (Escherichia coli; Pseudomonas aeruginosa; Proteus mirabilis; Micrococcus luteus; Bacillus subtilis); Fungi (Candida albicans; Candida tropicalis; Candida guilliermondii; Candida krusei); AIDS Virus HIV-I.

The uterine endometrial biopsy specimen of cadaver goat taken after implant of the IUCD of the present invention comprising polymer based material confirmed that the endometrium also becomes antimicrobial against the gram positive and gram negative bacteria and fungi against which the polymer based material has been tested for antimicrobial activity.

At present, the inventor has not been able to carry out in-vivo tests for AIDS HIV-I related antimicrobial action in the biopsy tissue. Nevertheless, the effects on bacteria and fungi strains is adequate proof of antimicrobial action developed in the endometrial tissue after the implantation of the IUCD comprising polymer based material of present invention in the uterus. In the live goat after implantation of IUCD comprising polymer based material of present invention only a very small sized endometrial biopsy was permissible. This tissue also tested for antimicrobial action against the bacteria Escherichia coli and fungus Candida albicans.

As can be seen, the preliminary studies on IUCD of present invention clearly and unambiguously indicate that the polymer based material in the uterus releases fragments of the polymer mass which via the cervical canal enters the vagina. The vagina, therefore, acquires antimicrobial action. Therefore, the possibility of the IUCD of present invention providing antimicrobial action in the vagina is also confirmed.

It may be noted that the IUCD of present invention can be removed from the uterine cavity without requiring invasive means to restore fertility by means of pulling means and flushing the uterine cavity with an alkaline solvent such as 10% Sodium bicarbonate solution As can be observed no stress in uterine cavity and/or on the uterine wall is caused during the entire procedure.

1. An improved intra-uterine contraceptive device (IUCD) comprising a combination of:
polymer based material 1, and
flexible structure 2,
characterized in that
the polymer based material 1 comprises a contraceptive polymer composition; and
the flexible structure 2 comprises
two or more of tubelets 3,
wherein the tubelets 3 are interconnected by connecting means 4,
wherein the tubelets 3 are provided with a pulling means 5 on one end,
wherein the tubelets 3 have perforations in the form of holes 6,
wherein the tubelets 3 have both ends 7, 8 open, and
wherein said ends 7, 8 are in sloping shape, and
wherein the tubelets 3 are arranged in the form of a chain, and
wherein the combination of said polymer based material 1 and said flexible structure 2 is injectable or implantable in the uterine cavity.

2-3. (canceled)

4. A device as claimed in claim 1, wherein said polymer based material is one which causes contraceptive effects.

5. A device as claimed in claim 1, wherein said polymer based material is one which cause contraceptive effects and antimicrobial effects.

6-8. (canceled)

9. A device as claimed in claim 1, wherein said flexible structure with perforated small tubelets is a free-form structure.

10. A device as claimed in claim 1, wherein said connecting means are flexible.

11. A device as claimed in claim 1, wherein said polymer based material 1 comprises contraceptive polymer composition, and a compound selected from the group comprising copper, radio-opaque material, or combination thereof.

12. A device as claimed in claim 11, wherein said copper is in the form of closed copper tubelets.

13-15. (canceled)

16. A device as claimed in claim 11, wherein said radio-opaque material is selected from barium sulfate and iodine or combination thereof.

17. (canceled)

18. A device as claimed in claim 1, wherein said contraceptive polymer composition is selected from a group comprising:
a) contraceptive polymer composition comprising high molecular weight styrene maleic anhydride and dimethyl sulfoxide (DMSO);
b) contraceptive polymer composition comprising a contraceptive polymer, a solvent medium, an electrically conducting material and a magnetic material; and
c) contraceptive polymer composition comprising styrene maleic anhydride based synergistic formulation comprising styrene maleic anhydride copolymer having lower molecular weight and styrene maleic anhydride copolymer having higher molecular weight dissolved in DMSO.

19. A device as claimed in claim 18, wherein in said contraceptive polymer comprising a contraceptive polymer, a solvent medium, an electrically conducting material and a magnetic material,
i) the contraceptive polymer is from the hydrogel class of polymers, particularly a mixture of styrene maleic anhydride copolymer and styrene maleic acid copolymer, and
ii) said solvent medium is dimethyl sulfoxide solvent, and
iii) said electrically conducting material is copper in its pure form essentially consisting of microsize particles and macrosize particles, and
iv) said magnetic material is iron in its pure form essentially consisting of microsize particles and macrosize particles.

20. A device as claimed in claim 18, wherein in said contraceptive polymer comprising said styrene maleic anhydride based formulation comprising styrene maleic anhydride (SMA) copolymer having lower molecular weight and styrene maleic anhydride (SMA) copolymer having higher molecular weight dissolved in DMSO,
i) the molecular weight of said SMA having lower molecular weight varies in the range from about 10000 to about 20000; and
ii) the molecular weight of said SMA having higher molecular weight varies in the range from about 60000 to about 100000.

21. A device as claimed in claim 1, wherein said polymer based material additionally comprises one or more of compounds selected from the group comprising spermicidal, ovicidal, antimicrobial, and antimicrobial fragment releasing compounds or combination thereof.

22. A device as claimed in claim 1, wherein said polymer based material additionally comprises one or more of compounds selected from the group comprising iron, copper, zinc and other materials or combination thereof to enhance structural strength and increase spermicidal, ovicidal and antimicrobial capabilities of said device.

23-25. (canceled)

26. An improved intra-uterine contraceptive device (IUCD) comprising a combination of:
polymer based material 1, and
flexible structure 2,
characterized in that
the polymer based material 1 comprises a contraceptive polymer composition; and
the flexible structure 2 comprises
two or more tubelets 3,
wherein the tubelets 3 are interconnected by connecting means 4,
wherein the tubelets 3 are provided with a pulling means 5 on one end,
wherein the tubelets 3 have perforations in the form of holes 6,
wherein the tubelets 3 have both ends 7, 8 open, and
wherein the said ends 7, 8 are in sloping shape, and
wherein the tubelets 3 are arranged in the form of a chain, and
wherein the combination of said polymer based material 1 and said flexible structure 2 is injectable or implantable in the uterine cavity; and
wherein said device is injectable or implantable in the uterus through an implantation means 9 comprising:
semi-rigid inserting means 10 provided with
i) a pushing means 11, and
ii) a syringe connector 12 having an inbuilt one way valve 13 which is capable of allowing the thick liquid from the syringe 14 to flow into the inserting means but obstruct back flow,
wherein the syringe 14 is provided with a pushing means (piston) 15, and
wherein the pushing means is connected to a thumb rest 16 with a connecting rod 17.

27-29. (canceled)

30. A device as claimed in claim 11, wherein said contraceptive polymer composition is selected from a group comprising:
a) contraceptive polymer composition comprising high molecular weight styrene maleic anhydride and dimethyl sulfoxide (DMSO);
b) contraceptive polymer composition comprising a contraceptive polymer, a solvent medium, an electrically conducting material and a magnetic material; and
c) contraceptive polymer composition comprising styrene maleic anhydride based synergistic formulation comprising styrene maleic anhydride copolymer having lower molecular weight and styrene maleic anhydride copolymer having higher molecular weight dissolved in DMSO.

31. A device as claimed in claim 21, wherein said contraceptive polymer composition is selected from a group comprising:
a) contraceptive polymer composition comprising high molecular weight styrene maleic anhydride and dimethyl sulfoxide (DMSO);
b) contraceptive polymer composition comprising a contraceptive polymer, a solvent medium, an electrically conducting material and a magnetic material; and
c) contraceptive polymer composition comprising styrene maleic anhydride based synergistic formulation comprising styrene maleic anhydride copolymer having lower molecular weight and styrene maleic anhydride copolymer having higher molecular weight dissolved in DMSO.

32. A device as claimed in claim 22, wherein said contraceptive polymer composition is selected from a group comprising:
a) contraceptive polymer composition comprising high molecular weight styrene maleic anhydride and dimethyl sulfoxide (DMSO);
b) contraceptive polymer composition comprising a contraceptive polymer, a solvent medium, an electrically conducting material and a magnetic material; and
c) contraceptive polymer composition comprising styrene maleic anhydride based synergistic formulation comprising styrene maleic anhydride copolymer having lower molecular weight and styrene maleic anhydride copolymer having higher molecular weight dissolved in DMSO.
